SEQUENCE LISTING (3 sequences)

SEQ ID NO: 1 - DNA, 17 bp, Unknown
  Primer
gaggcttccaggcgtcc

SEQ ID NO: 2 - DNA, 27 bp, unknown
  Primer
agtgatgaagtagttggtgaccgtctg

SEQ ID NO: 3 - DNA, 20 bp, Unknown
  Primer
ccttcttgctggcacgcaat

(19) United States
(12) Patent Application Publication
(10) Pub. No.: US 2004/0033524 A1
(43) Pub. Date: Feb. 19, 2004

(54) Beta-adrenoceptor genetic polymorphisms and obesity

(76) Inventor: Julie A. Johnson, Melrose, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/617,846

(22) Filed: Jul. 10, 2003

Related U.S. Application Data
(62) Division of application No. 10/075,490, filed on Feb. 12, 2002.
(60) Provisional application No. 60/268,310, filed on Feb. 13, 2001.
(60) Provisional application No. 60/269,096, filed on Feb. 14, 2001.

(51) Int. Cl. C12Q 1/68; A61K 31/137

(52) U.S. Cl. 435/6; 514/651

(57) ABSTRACT

The present invention provides a method for identifying a subject having a risk of developing obesity, coronary microvasular dysfunction, or hypertension, comprising detection of the presence of a single nucleotide polymorphism (SNP) in a nucleic acid encoding an element of at least one β-adrenergic receptor from the subject. The presence of the SNP is correlated with obesity, coronary microvascular dysfunction, or hypertension, and thereby identifies the subject as having a risk of developing obesity, coronary microvascular dysfunction, of hypertension. The subject invention also provides methods of identifying patients likely to benefit from the prescription of beta blocker hypertension medications. In various embodiments, the nucleic acids detected include those genes encoding ADRB1 (β₁AR), ADRB2 (β₂AR), ADRB3 (β₃AR), GNB3 (G protein β3 subunit), or GNAS1 (Gₛ protien alpha subunit). Methods of treating identified individuals are also provided.

### CROSS-REFERENCE TO RELATED APPLICATIONS

The present application is a divisional of U.S. patent application Ser. No. 10/075,490, filed Feb. 12, 2002, pending, which claims benefit of U.S. Provisional Application Serial No. 60/268,310, filed Feb. 13, 2001; and U.S. Provisional Application Serial No. 60/269,096, filed Feb. 14, 2001. Each of these applications is hereby incorporated by reference herein in their entireties, including any nucleic acid sequences, figures, tables, or drawings.

The subject invention was made with government support under a research project supported by the National Institutes of Health, Grant Number R01 HL 64691. The Government may have certain rights in this invention.

### BACKGROUND OF INVENTION

The prevalence of obesity has increased dramatically in Westernized societies. It represents a major health problem, because it markedly increases the risk of cardiovascular disease, diabetes, lipid disorders, and some cancers. Obesity clearly has a heritable basis, with estimates that 40% to 70% of the variability in body weight is genetically mediated. Thus, understanding the genetic basis of variability in BMI or obesity is important. The range of treatments for obesity reflects the complexity of the processes involved in weight regulation and the current lack of understanding of these processes. Recent reports have even implicated viruses as a possible causative factor in obesity (U.S. News and World Report, Aug. 7, 2000). In addition, human twin studies strongly suggest a substantial genetic basis in the control of body weight, with estimates of heritability of 80% to 90% (Simopoulos, A. P. and Childs, B., eds. [1989] “Genetic Variation and Nutrition in Obesity”, World Review of Nutrition and Diabetes, 63, S. Karger, Basel, Switzerland; Boijeson, M. [1976] Acta. Paediatr. Scand. 65:279-287).

The regulation of body weight, and particularly body fat, in animals is a complex process having enormous implications for the health and well being of humans and other animals. Excess body weight and/or an excess of body fat relative to lean body mass has been associated with a wide range of health problems. Obesity is a major health problem in the United States, with estimates that 50% to 60% of Americans over age 30 are overweight and 25% to 30% are clinically obese (Wickelgren, L. [1998] “Obesity: How Big a Problem?” [news], Science 280(5368):1364-7). Obesity is of concern because numerous studies link obesity with increased risk of cardiovascular disease, metabolic disorders (such as Type II diabetes mellitus and lipid abnormalities) and some forms of cancer.

Individuals 20% over ideal weight guidelines are considered obese. Obesity is classified as mild (20-40% overweight), moderate (41-100% overweight), and severe (>100%). Severe obesity is relatively rare, affecting less than 0.5% of all obese individuals and about 0.1 % of the total population.

Obesity in humans is treated by a variety of means ranging from surgical procedures (gastric bypass) for the severely obese to diet therapy, behavior modification, and medication for the mildly to moderately obese. Management of moderate and mild obesity is typically performed by the individual and commercial organizations that provide behavior modification programs and, in some cases, prepackaged diets. The medical community recommends that diet treatments be administered under medical supervision.

Approximately 50 million (or about 1 in 4 adult) Americans have high blood pressure. Currently, treatment of high blood pressure (hypertension) is by a trial and error approach and consensus treatment guidelines recommend beta-blockers or diuretics as preferred first line therapy (unless patients have compelling indications for another first line therapy). The trial and error approach has numerous problems because in any given patient, the likelihood of response to a specific blood pressure lowering medicine, including beta-blockers, is only 40-70%. This means that if 100 people were given a beta-blocker for their high blood pressure, between 40 and 70 would have adequate lowering of their blood pressure. As a result, patients can have poor control of their blood pressure for extended periods of time as attempts are made to find the right medication.

Additionally, because hypertension is a silent disease (meaning it does not, typically, cause any symptoms), patients often become quickly disenfranchized with the health care system if a medication to control their blood pressure isn't quickly found. There are currently no easy methods for identifying individuals most likely to respond to a beta-blocker (or other blood pressure medication). The present invention provides a genetic test that can be used to identify those individuals most likely to have a good blood pressure response to beta-blocker therapy.

### BRIEF DESCRIPTION OF THE INVENTION

The present invention provides a method for identifying a subject having a risk of developing obesity, coronary microvascular dysfunction, or hypertension, comprising detection of the presence of a single nucleotide polymorphism (SNP) in a nucleic acid encoding an element of at least one β-adrenergic receptor from the subject. The presence of the SNP is correlated with obesity, coronary microvascular dysfunction, or hypertension, thereby identifying the subject as having a risk of developing obesity, coronary microvascular dysfunction, or hypertension. In various embodiments, the nucleic acids detected include those genes encoding ADRB1 (β₁AR), ADRB2 (β₂AR), ADRB3 (β₃AR), GNB3 (G protein β3 subunit), and/or GNAS1 (Gₛ protein alpha subunit). Methods of treating identified individuals are also provided.

The present invention also provides a method for identifying an allele correlated with obesity, coronary microvascular dysfunction or hypertension, comprising detection of the presence of a single nucleotide polymorphism (SNP) in a nucleic acid encoding at least one component of a β-adrenergic receptor from a subject, wherein the presence of the SNP is correlated with obesity, coronary microvascular dysfunction, or hypertension. This identifies the allele as being correlated with obesity, coronary microvascular dysfunction, or hypertension. In various embodiments, the nucleic acids detected include those genes encoding ADRB1 (β₁AR), ADRB2 (β₂AR), ADRB3 (β₃AR), GNB3 (G protein β3 subunit), and/or GNAS1 (Gₛ protein alpha subunit). Methods of treating identified individuals are also provided.

In another aspect of the invention, people with a certain form of the beta-adrenergic receptor are more likely to have a good blood pressure response to beta-blockers than individuals with another form of the beta-adrenergic receptor. Thus, the subject invention includes tests that allow for individualization or personalization of drug therapy, thus reducing the trial and error approach in the treatment of hypertension. Specifically, a patient is genotyped prior to receiving a prescription for their blood pressure. Depending upon genotype, patients are given a prescription for a beta-blocker or an alternative blood pressure medication. This should allow for more precise prescribing, will shorten the time that patients have blood pressure out of control, and will minimize patients becoming disenfranchized with the health care system because the right medication is found quickly.

### BRIEF DESCRIPTION OF THE TABLES

Table 1 provides the polymorphisms and allelic frequencies (of the least common allele) of three β-adrenergic receptors.

Table 2 illustrates the association of β₁1AR codon 49 and obesity.

Table 3 shows the demographic characteristics and 24-hr blood pressures of dippers and non-dippers.

Table 4 depicts β₂-adrenergic receptor allele frequencies for nighttime BP dippers and non-dippers.

Table 5 provides the allelic frequencies of responders and non-responders to beta blocker medications.

Table 6 illustrates the blood pressure response based upon the codon 389 genotype.

Table 7 depicts the blood pressure response based upon the codon 49 genotype.

### BRIEF DESCRIPTION OF THE FIGURES

FIG. 1 illustrates the observed systolic (SBP) and diastolic (DBP) blood pressure declines from day to night in the non-dipper group (black bars) and in the dipper group (white bars).

### DETAILED DISCLOSURE

The present invention provides a method for identifying a subject having a risk of developing obesity, coronary microvascular dysfunction or hypertension, comprising detection of the presence of a single nucleotide polymorphism (SNP) in a nucleic acid encoding at least one β-adrenergic receptor from the subject. The presence of the SNP is correlated with obesity, coronary microvascular dysfunction, or hypertension, thereby identifying the subject as having a risk of developing obesity, coronary microvascular dysfunction, or hypertension. In various embodiments, the nucleic acids detected include those genes encoding ADRB1 (β₁AR), ADRB2 (β₂AR), ADRB3 (β₃AR), GNB3 (G protein β3 subunit), or GNAS1 (Gₛ protein alpha subunit).

The present invention also provides a method for identifying an allele correlated with obesity, coronary microvascular dysfunction, or hypertension, comprising detection of the presence of a single nucleotide polymorphism (SNP) in a nucleic acid encoding at least one component of a β-adrenergic receptor from a subject, wherein the presence of the SNP is correlated with obesity, coronary microvascular dysfunction, or hypertension. This identifies the allele as being correlated with obesity, coronary microvascular dysfunction, or hypertension. In various embodiments, the nucleic acids detected include those genes encoding ADRB1 (β₁AR), ADRB2 (β₂AR), ADRB3 (β₃AR), GNB3 (G protein β3 subunit), or GNAS1 (Gₛ protein alpha subunit).

The presence of an SNP is identified by determining the nucleic acid sequence of at least a region of a gene encoding a β-adrenergic receptor, or receptor associated protein, according to standard molecular biology protocols well known in the art, for example, as described in Sambrook et al. (1989) or as set forth in the Examples provided herein. One skilled in the art will appreciate that the gene or a region thereof can contain one or more SNP's associated with obesity and/or diabetes. Examples of methods of nucleic acid detection known in the art, such as nucleic acid sequencing, polymerase chain reaction (PCR) with or without restriction fragment length polymorphism (RFLP) analysis, Southern and Northern blot analysis, ligase chain reaction, and PCR reaction of specific alleles (PASA) can be utilized to enhance the subject assay and are described for example in Sambrook et al.

Other techniques such as isothermal, single-cycle amplification technique, termed the self-sustained sequence replication (3SR) system, can discriminate between a wild-type or mutant sequence at any particular residue. Because this amplification method generates a predominance of one strand of single-stranded RNA, direct sequencing is possible.

One method for detecting a nucleic acid encoding at least one β-adrenergic receptor variant or a variant receptor associated protein can comprise contacting the pre-selected portion of a whole blood sample with at least one detectable nucleic acid probe that is selective for the nucleic acid encoding at least one β-adrenergic receptor variant or a variant receptor associated protein under conditions favorable for promoting hybridization of the probe. The method then detects the presence of the hybridization between the probe and the nucleic acid, thereby detecting the presence of the nucleic acid encoding at least one β-adrenergic receptor variant or a variant receptor associated protein. Conditions favorable for promoting hybridization of a particular probe to a nucleic acid can vary depending upon the sequence of the nucleic acid being detected or the type of probe utilized. However, such conditions are generally known in the art and will be apparent to the skilled artisan. Thus, one can merely adapt the procedures set forth in the art to suit the present methods.

In some embodiments, the invention further provides for the treatment of individuals identified as being at risk for the development of obesity, hypertension, or coronary microvascular dysfunction. In this aspect of the invention, individuals identified as being at risk of the development of obesity can be treated by a variety of means including surgical procedures (e.g., gastric bypass), diet therapy, behavior modification, counseling, and medications well-known to the skilled practioner. For the treatment of coronary microvascular disease, individuals can be treated by counseling, education as to behaviors that increase the risk of microvascular diseases (such as smoking, anger management, etc.), plasmapheresis, behavior modification, and/or corticosteroids. Hypertension can be treated by any art-recognized method, including the use of anti-hypertensives.

The subject invention also provides methods for predicting the responsiveness of an individual to beta-blocker medications comprising genotyping the β₁ adrenergic receptor (β₁AR) of said individual at codon 49, at codon 389, or at both codon 49 and 389. After the individual has been genotyped, the receptor phenotype is observed and recorded. Because the presence of a Ser49, Arg 389, or Ser49/Arg389 phenotype is indicative of a likely response to said beta-blocker medication, individuals having such phenotypes can be expected to respond to treatment with beta blockers and such treatment can be initiated. Those patients not having the aforementioned phenotypes can be treated using alternative anti-hypertensive agents such as those listed below.

Beta blocker medications typically used in modulation/control of hypertension are, typically, selected from the group consisting of acebutolol, atenolol, betaxolol, bisoprolol, esmolol, metoprolol, long-acting metoprolol, carteolol, nadolol, penbutolol, pindolol, propranolol, long-acting propranolol, sotalol, timolol, labetalol, and salts thereof. Alternatively, and combinations of one or more beta blocker can be used by the skilled practioner in the treatment regimen. In other embodiments, where an individual response to beta blocker therapy is observed, however the lowering of blood pressure is insufficient to consider the condition controlled, other medications can be added to the therapeutic regimen to control blood pressure.

Where beta blocker medications fail to adequately modulate or control the blood pressure of an individual, alternative anti-hypertension medications such as diuretics, angiotensin converting enzyme (ACE) inhibitors, or calcium antagonists, or salts thereof can be used. Combinations of the aforementioned alternative medications can also be used by the skilled practioner, optionally in conjunction with beta blocker medication. These alternative anti-hypertensive medications can also be used for those individuals not expected, on the basis of phenotype, to be responsive to beta blocker therapy.

Diuretics include, and are not limited to, bendroflumethiazide, benzthiazide, chlorothiazide, cyclothiazide, hydrochlorothiazide, hydroflumethiazide, indapamide, methyldlothiazide, metolazone, polythiazide, quinethazone, trichlormethiazide, bumetanide, ethacrynic acid, furosemide, amiloride, spironolactone, triamterene. ACE inhibitors include, and are not limited to, captopril, enalapril, fosinopril, lisinopril, ramipril, and salts thereof. Calcium antagonists include, and are not limited to, diltiazem, sustained release diltiazem, verapamil, sustained release verapamil, nifedipine (sustained release), nifedipine, nicardipine, isradipine, nimodipine and salts thereof.

Thus, the subject invention also provides methods of reducing delays in blood pressure control in an individual comprising:

a) genotyping:

1) the β₁ adrenergic receptor (β₁AR) of said individual at codon 49, wherein the presence of the Ser49 phenotype is indicative of a likely response to said beta-blocker medication;

2) the β₁ adrenergic receptor (β₁AR) of said individual at codon 389, wherein the presence of the Arg389 phenotype is indicative of a likely response to said beta-blocker medication; or

3) the β₁ adrenergic receptor (β₁AR)) of said individual at codons 49 and 389, wherein the presence of the Ser49/Arg389 phenotype is indicative of a likely response to said beta-blocker medication; and

b) providing, on the basis of the observed phenotype, an appropriate anti-hypertensive agent, wherein beta blocker medications are prescribed to an individual having a Ser49 phenotype, Arg389 phenotype, or a Ser49/Arg389 phenotype and wherein patients lacking a Ser49 phenotype, Arg389 phenotype, or a Ser49/Arg389 phenotype are prescribed alternative non-beta blocker antihypertensive medications.

It should be understood that the embodiments and Examples described herein are for illustrative purposes only and that various modifications or changes in light thereof will be suggested to persons skilled in the art and are to be included within the spirit and purview of this application. All patents, patent applications, provisional applications, and publications referred to or cited herein are incorporated by reference in their entirety to the extent they are not inconsistent with the explicit teachings of this specification.

### EXAMPLE 1

ADRB1 (β₁AR gene) and Obesity

β₁AR genotypes at codons 49 and 389 were determined by PCR and restriction fragment length polymorphism (RFLP) analysis using standard techniques. A total of 89 WISE participants were genotyped at codon 49; 61 WISE participants were also genotyped at codon 389. Data for codon 49 are shown in Table 2 below.

In vitro and human studies provide evidence that the β₁AR polymorphisms are functional. Genotyping of 89 WISE participants at codon 49 of the β₁AR revealed significant differences in BMI between Ser49Ser and Gly49 carriers. The data indicates an association exists between the β₁AR polymorphism and obesity.

### EXAMPLE 2

ADRB2 (β₁AR gene) and Obesity

Analysis was also performed regarding of the β₂AR polymorphisms and obesity in the WISE population. A total of 34 patients were genotyped for the three β₂AR polymorphisms. The Glu27 allele is associated with a higher BMI. Mean BMI in the Glu27 carriers is 31.7 kg/m², versus 28.8 kg/m² in the Gln27Gln group.

### EXAMPLE 3

ADRB2 (β₂AR gene) and Hypertension/Coronary Dysfunction

Polymorphisms of the β₂AR were analyzed by haplotype and revealed associations between dipper phenotype in hypertension and β₂AR gene. This analysis also revealed a correlation between coronary microvascular dysfunction and the β₂AR gene. In these analyses, three chromosomal haplotypes were found: Arg19/Gly 16/Glu27 (RGE), Cys19/Gly16/Gln27 (CGQ), and Cys19/Arg16/Gln27 (CRQ). Data from both studies suggests the RGE haplotype is detrimental (significantly associated with the undesirable phenotype), CGQ is protective (significantly associated with the desirable phenotype), and CRQ is neutral. These analyses were performed using standard techniques as described below.

Thirty-nine unrelated, previously diagnosed hypertensive patients, ages 35 to 65 years, were enrolled in the study. After obtaining written informed consent, a blood sample was drawn from each patient for determination of the β₂AR genotype. Any current anti-hypertensive therapy was discontinued ≧2 weeks prior to ambulatory BP monitoring. Hypertension, defined as an average of two sitting diastolic BP readings >90 mm Hg after ≧5 minutes of rest, was confirmed in all patients on two occasions ≧2 weeks after discontinuing antihypertensive drug therapy. A 24-hour ausculatory BP monitor (Accutracker II™ or Accutracker DX,™ Suntech Medical Instruments, Raleigh, N.C.), programmed to measure BP four times per hour from 0600 to 2300 and three times per hour from 2300 to 0600, was placed on the patient's non-dominant arm. To ensure accuracy, blood pressures from the ambulatory BP monitor were compared to those obtained with a mercury sphygmomanometer on the opposite arm. The study was started when both systolic BP and diastolic BP measurements by the two methods were within 5 mm Hg of each other. The patient was instructed to resume usual daily activities and return in 24 to 25 hours for monitor removal.

Genomic DNA was extracted from lymphocytes (Puregene® DNA Isolation Kit, Gentra Systems, Inc., Minneapolis, Minn.) and used to generate a 318 bp PCR product with primers (forward) 5′-GAGGCTTCCAGGCGTCC-3′ (SEQ ID NO: 1) and (reverse) 5′-AGTGATGAAGTAGTTGGTGACCGTCTG-3′ (SEQ ID NO: 2). Each 50 μl PCR reaction contained 50-200 ng genomic DNA in standard conditions as recommended by the Taq polymerase manufacturer (Promega, Madison Wis.). PCR was performed on a GeneAmp® PCR System 9600 Thermal Cycler (Applied Biosystems, Foster City, Calif.) as follows: denaturation at 94° C. for 5 min, followed by 35 cycles of 94° C. for 30 s, 61° C. for 30 s, and 72° C. for 30 s, and a final 10 min extension at 72° C. To determine genotype at codon 5′LC-19, the 318 bp PCR product was digested with MspA I (New England Biolabs, Inc., Beverly, Mass.), separated on an 8% polyacrylamide gel, and visualized with ethidium bromide staining. The 5′LC-Cys19/Cys genotype remained undigested (318 bp). Fragment sizes of 54 bp and 264 bp were produced for the 5′LC-Arg19/Arg genotype, and 54 bp, 264 bp, and 318 bp fragments were produced for the heterozygous genotype.

The 318 bp PCR product was used as a template to generate a 194 bp PCR product for determination of codon 16 and 27 genotypes. The PCR was carried out as described above except that an internal forward primer (5′-CCTTCTTGCTGGCACGCAAT-3′ (SEQ ID NO: 3)) that differed from the β₂AR gene sequence by one nucleotide was used to create a restriction site for determination of the codon 16 genotype. Digestion of the 194 bp PCR product with BsrDI (New England Biolabs) yielded fragments of 12, 24, 51, and 107 bp for the Gly16/Gly genotype; 12, 51, and 131 bp for the Arg16/Arg genotype; and 12, 24, 51, 107, and 131 bp for the Arg16/Gly genotype. In a separate reaction, the 194 bp PCR product was digested with BbvI (New England Biolabs) to determine codon 27 genotype. The Glu27/Glu27 genotype remains undigested (194 bp). Fragments of 62 bp and 132 bp were produced for the homozygous Gln27 genotype, and 62, 132, and 194 bp fragments were produced for the Gln27/Glu genotype.

For heterozygotes at two or three positions, haplotype could not be inferred, and thus was determined experimentally. The 318 bp PCR products were purified (QIAquick® PCR Purification Kit, QIAGEN, Inc., Valencia, Calif.) and cloned into a pGEM®-T vector system (Promega). Recombinant colonies were screened for β₂AR gene inserts by PCR as described for the 318 bp PCR product. Purified PCR products were sequenced using ABI PRISM® BigDye™ Terminator cycle sequencing protocols (Applied Biosystems, Foster City, Calif.) and sequencing primer 5′-AGTGATGAAGTAGTTGGTGACCGTCTG-3′ (SEQ ID NO: 2). Each clone represented one of the two gene copies, and haplotypes were thus determined directly from the sequence.

Ambulatory BP data were evaluated by standard criteria as previously described (Johnson et al. [1995] Am. J. Hypertens. 8:254-259). An ambulatory BP profile was considered valid when at least 75% of the measurements were usable. Hourly BP averages were calculated from which mean daytime (1000 to 2000) and nighttime (2400 to 0600) systolic and diastolic blood pressures were determined for each patient. Daytime and nighttime were defined using narrow fixed time intervals as described by Verdecchia (Hypertension [2000] 35:844-851). As in previous studies, dippers and non-dippers were defined as those with ≧10% and <10% decline in mean daytime to nighttime systolic BP, respectively. Baseline characteristics were compared between dippers and non-dippers by Chi-square analysis for nominal data and Student's unpaired t-test for continuous data. Chi-square and Fisher's exact tests were conducted to compare haplotype frequency between dippers and non-dippers. Odds ratios and 95% confidence intervals were determined for the association of dipper/non-dipper phenotype with β₂AR haplotype.

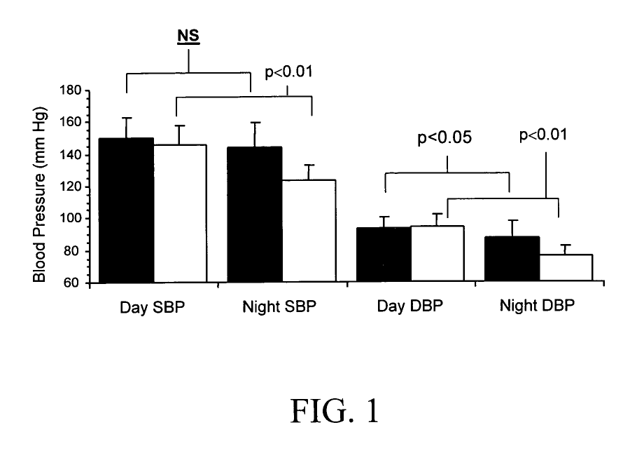

There were no significant differences in demographic characteristics or daytime blood pressures between the two groups (Table 3). Systolic and diastolic blood pressures declined significantly from day to night in the dipper group, but only diastolic BP declined significantly in the non-dipper group (FIG. 1). The mean decline in systolic BP from day to night was 14.8±3.8% for dippers and 3.8±5.5% for non-dippers. The mean nocturnal diastolic BP decline was 19.6±5.4% for dippers and 6.3±7.4% for non-dippers.

Experiments revealed only three β₂AR haplotypes: 5′LC-Arg19/Gly16/Glu27 (RGE), 5′LC-Cys19/Arg16/Gln27 (CRQ), and 5′LC/Cys19/Gly16/Gln27 (CGQ). The β2adrenergic receptor haplotype frequency differed significantly between the dipper and non-dipper groups (p=0.035 by χ² analysis) as shown in Table 4. Specifically, allele frequencies of the RGE haplotype were 42.5% for non-dippers and 18.4% for dippers (p=0.02). Of the 21 patients carrying the RGE haplotype, 14 (67%) were non-dippers. The OR for the association of the RGE haplotype with the non-dipper phenotype versus the CGQ haplotype was 5.3 (95% confidence interval 1.4 to 19.5). The OR for association of the RGE haplotype with the non-dipper phenotype versus any other haplotype (CRQ or CGQ) was 3.3 (95% confidence interval 1.2 to 9.2). The CGQ allele frequency was greater in the dipper versus the non-dipper group (p=0.048). The CRQ haplotype was not associated with the dipper/non-dipper phenotype.

The homozygous β₂AR genotype was significantly associated with dipper status (p=0.04 by Fisher's exact test). Only three patients were homozygous for the RGE haplotype, all of whom were non-dippers. The homozygous CGQ genotype occurred only in dippers, and the homozygous CRQ genotype was similarly distributed between groups.

### EXAMPLE 4

Responsiveness to Beta Blocker Therapy

Beta-blockers are drugs commonly used in the treatment of hypertension (high blood pressure). They are recommended as preferred initial therapy by consensus guidelines. Approximately 30-60% of patients prescribed a beta-blocker for their hypertension will not have an adequate reduction in blood pressure.

Genetic variation (polymorphisms) in the gene encoding the beta 1-adrenergic receptor (β₁AR) contribute to the variability in response to beta-blockers. There are two common, non-synonymous polymorphisms of the P AR gene and they result in either a serine (Ser) or glycine (Gly) at codon 49, and arginine (Arg) or glycine (Gly) at codon 389. The relationship between these polymorphisms and heart rate and blood pressure response to the beta-blocker metoprolol was tested in 28 individuals with hypertension.

Patients were washed out from all their antihypertensive medications for at least 2 weeks. They then underwent baseline testing which included recording of 24-hour ambulatory blood pressure data and treadmill exercise heart rate responses at 10 AM and 2 PM. Patients were started on metoprolol and titrated to a dose that produced a diastolic blood pressure DBP<90 mm Hg, or until they reached the maximum dose (200 mg twice daily). Patients were maintained on this target dose for at least 4 weeks and then underwent repeat studies, as at baseline.

It was found that the heart rate response to beta-blockade with metoprolol was not associated with genotype. Daytime, but not nighttime, blood pressure response to metoprolol was significantly greater in Arg389 homozygotes, as compared to carriers of the Gly389 form of the receptor. In contrast, change in nighttime blood pressure was significantly greater in Ser49 homozygotes as compared to carriers of the Gly49 allele, and differences in response for daytime blood pressure were of borderline significance. Patients were also divided as responders or non-responders, with non-responders defined as those with a <10% decline in DBP, and compared allele frequencies in these groups.

While the allele frequencies were not different between the responders and non-responders for the codon 49 allele, there were highly significant differences with the codon responders. The data provides compelling evidence that the genotype at codon 389 of the β₁AR is a strong predictor of blood pressure response to beta-blocker therapy, and the codon 49 genotype provides additional, information about response.

As described above, the current approach to treating hypertension is by trial and error. Many patients require trials on several medications before finding the right one. This technology would allow for more personalized prescribing, based on the patient's genetic make-up. The benefits of this could be numerous.

TABLE 1
Allele freq of LEAST common allele

| | Allele | Blacks | Whites |
|---|---|---|---|
| ADRB 1 | | | |
| Ser49Gly | Gly49 | 0.13 | 0.15 |
| Arg389Gly | G1y389 | 0.42 | 0.27 |
| ADRB2 | | | |
| 5′LCCys19Arg | Arg19 | 0.21 | 0.35 |
| Arg16Gly | Arg16 | 0.49 | 0.46 |
| Gln27Glu | Glu27 | 0.21 | 0.35 |
| ADRB3 | | | |
| Trp64Arg | Arg64 | ? | 0.07 |
| GNB3 | | | |
| C825T | 825T | 0.79 | 0.33 |
| GNAS1 | | | |
| Cdnl31 T→C | FokI-(T) | ? | 0.47 |
| Cdn371 C→T | FokI-(T) | 0.22 | 0.05 |

TABLE 2
Association of β₁1AR Codon 49 Genotype & Obesity

| | Ser49Ser | Gly49 carriers | |
|---|---|---|---|
| | n = 65 | n = 24 | P |
| Weight (kg) | 85 + 19 | 76 + 14 | 0.02 |
| BMI (kg/m²) | 32.8 + 7 | 28.5 + 5 | 0.004 |
| WHR | 0.85 + 0.09 | 0.89 + 0.14 | NS |
| Obese^{a} (%) | 62% | 38% | 0.04 |

Mean + SD: ^{a}Obese defined as BMI > 30 kg/m²

TABLE 3
Demographic characteristics and
24-hr blood pressures of dippers and non-dippers

| Characteristic | Dippers (n = 19) | Non-dippers (n = 20) |
|---|---|---|
| Age (years) | 45 ± 6 | 50 ± 10 |
| Race (Caucasian/African | 12/7 | 10/10 |
| American) | | |
| Gender (male/female) | 11/8 | 10/10 |
| Body Mass Index (kg/m²) | 27 ± 5 | 30 ± 5 |
| No. of patients with diabetes | 0 | 0 |
| mellitus | | |
| No. of current smokers | 2 | 2 |
| Daytime Systolic BP | 146 ± 12 | 150 ± 13 |
| (mm Hg) | | |
| Daytime Diastolic BP | 94 ± 8 | 93 ± 7 |
| (mm Hg) | | |

Mean ± SD

TABLE 4
β₂-adrenergic receptor allele frequencies
for nighttime BP dippers and non-dippers

| | Dippers | Non-dippers | p | Odds | |
|---|---|---|---|---|---|
| Allele | (n = 38 alleles) | (n = 40 alleles) | value* | Ratio | 95% CI |
| RGE | 7 (18.4%) ^{†} | 17 (42.5%) | | 5.3 | (1.4-19.5) |
| CRQ | 18 (47.4%) | 17 (42.5%) | 0.035 | 2.0 | (0.6-6.6) |
| CGQ | 13 (34.2%) | 6 (15.0%) | | 1 | |

RGE = 5′LC-Arg19/Gly16/Glu27; CRQ = 5′LC-Cys19/Arg16/Gln27; CGQ = 5′LC-Cys19/Gly16/Gln27
*p value determined by χ² analysis of 3 × 2 contingency table
^{†}Number of alleles (%)

TABLE 5
β₁AR Allele Frequencies In Responders And Non-responders

| | Responders | Non-responders | |
|---|---|---|---|
| Codon 389 allele | | | |
| Arg 389 | 87.5% | 57.1% | |
| Gly 389 | 12.5% | 42.9% | p < 0.001 |
| Codon 49 allele | | | |
| Ser49 | 87.5% | 75% | |
| Gly49 | 12.5% | 25% | NS |

P by Chi² analysis

TABLE 6
Blood pressure response (% change) based on codon 389 genotype

| Response | Arg389Arg | Gly carriers | p |
|---|---|---|---|
| 24 hr SBP | −10.5 ± 7.9 | −3.3 ± 12.6 | 0.075 |
| 24 hr DBP | −12.6 ± 7.4 | −4.6 ± 8.3 | 0.013 |
| Daytime SBP | −11.6 ± 8.0 | −2.8 ± 12.5 | 0.033 |
| Daytime DBP | −13.6 ± 7.2 | −3.5 ± 8.7 | 0.0025 |
| Nighttime SBP | −7.5 ± 9.5 | −3.9 ± 13.9 | NS |
| Nighttime DBP | −10.6 ± 11.6 | −7.3 ± 9.1 | NS |

p by t-test
SBP = systolic blood pressure; DBP = diastolic blood pressure

TABLE 7
Blood pressure response (% change) based on codon 49 genotype

| Response | Ser49Ser | Gly carriers | p |
|---|---|---|---|
| 24 hr SBP | −10.1 ± 9.1 | −0.6 ± 12.2 | 0.04 |
| 24 hr DBP | −10.7 ± 7.8 | −3.9 ± 8.9 | 0.052 |
| Daytime SBP | −10.2 ± 9.3 | −1.0 ± 12.8 | 0.058 |
| Daytime DBP | −10.3 ± 8.2 | −4.0 ± 10.4 | 0.107 |
| Nighttime SBP | −9.3 ± 10.9 | +0.6 ± 11.8 | 0.036 |
| Nighttime DBP | −12.4 ± 9.4 | −3.1 ± 9.0 | 0.016 |

p by t-test
SBP = systolic blood pressure; DBP = diastolic blood pressure

References

Waltman, C., et al. [1994] “Polymorphism of the Gene Encoding the Alpha Subunit of the Stimulatory G-Protein of Adenylyl Cyclase (GNAS1)” Hum Genet 93(4):477-8.

Jla, H., et al. [ 1999] “Association of the G(s)α Gene with Essential Hypertension and Response to β-Blockade,” Hypertension 34(1):8-14.

Moore, J. D., et al., “Racial Differences in the Frequencies of Cardiac β(1)-Adrenergic Receptor Polymorphisms: Analysis of cI45A>G and c1165G>C,” Hum Mutat (Online) 14(3):271.

Sirleft, W., et al. [1999] “Worldwide Ethnic Distribution of the G Protein β3 Subunit 825T Allele and its Association with Obesity in Caucasian, Chinese, and Black African Individuals,” J Am. Soc. Nephrol. 10(9):1921-30.

Xie, H. G., et al. [1999] “Interethnic Differences in β2 Adrenoceptor Polymorphisms in African-American, Caucasian and Chinese Populations” (abstract), Clin. Pharmacol. Ther. 65(2):133.

1. A method of reducing delays in blood pressure control in an individual comprising:
a) genotyping:
1) the β₁ adrenergic receptor (β₁AR) of said individual at codon 49, wherein the presence of the Ser49 phenotype is indicative of a likely response to said beta-blocker medication;
2) the β₁ adrenergic receptor (β₁AR) of said individual at codon 389, wherein the presence of the Arg389 phenotype is indicative of a likely response to said beta-blocker medication; or
3) the β₁ adrenergic receptor (β₁AR)) of said individual at codons 49 and 389, wherein the presence of the Ser49 and Arg389 phenotype is indicative of a likely response to said beta-blocker medication; and
b) providing, on the basis of the observed phenotype, an appropriate anti-hypertensive agent, wherein beta blocker medications are prescribed to an individual having a Ser49 phenotype, Arg389 phenotype, or a Ser49/Arg389 phenotype and wherein patients lacking a Ser49 phenotype, Arg389 phenotype, or a Ser49 and Arg389 phenotype are prescribed alternative non-beta blocker antihypertensive medications.

2. The method according to claim 1, wherein said beta blocker medication is selected from the group consisting of acebutolol, atenolol, betaxolol, bisoprolol, esmolol, metoprolol, long-acting metoprolol, carteolol, nadolol, penbutolol, pindolol, propranolol, long-acting propranolol, sotalol, timolol, labetalol, salts thereof, and combinations thereof.
